(19) United States
(12) Patent Application Publication
(10) Pub. No.: US 2009/0136522 A1
(43) Pub. Date: May 28, 2009

(54) Multivalent Immunogen

(76) Inventors: Barton F. Haynes, Durham, NC (US); Michael Moody, Durham, NC (US); Laurent Verkozcy, Durham, NC (US); Bruce A. Sullenger, Durham, NC (US); Juliana Layzer, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/225,541

(22) PCT Filed: Mar. 26, 2007
(86) PCT No.: PCT/US2007/007399

§ 371 Date: Sep. 24, 2008

Related U.S. Application Data
(60) Provisional application No. 60/785,376, filed on Mar. 24, 2006.

(51) Int. Cl. A61K 39/395; C07K 14/00; C07K 7/00; A61K 39/12

(52) U.S. Cl. 424/173.1; 530/326; 530/327; 530/324; 530/367; 424/196.11

(57) ABSTRACT

The present invention relates, in general, to HIV and, in particular, to immunogens that present epitopes located in the membrane external proximal region (MPER) of HIV-I envelope gp41 in multivalent form and to methods of using same.

This application claims priority from U.S. Provisional Application No. 60/785,376, filed Mar. 24, 2006, the entire content of which is incorporated herein by reference.

### TECHNICAL FIELD

The present invention relates, in general, to HIV and, in particular, to immunogens that present epitopes located in the membrane external proximal region (MPER) of HIV-1 envelope gp41 in multivalent form and to methods of using same.

### BACKGROUND

The dearth of broadly neutralizing antibodies in acute or early infection and in response to vaccination with HIV-1 envelope is a major issue haunting the AIDS research field. Two key neutralizing anti-HIV-1 monoclonal antibodies (mAbs), 2F5 and 4E10, bind to epitopes that lie in the membrane external proximal region (MPER) of HIV-1 envelope gp41 (FIG. 1) (Muster et al, J. Virol. 67:6642 (1993); Steigler et al, AIDS Research & Human Retroviruses 17:1757 (2001); Zwick et al, J. Virol. 75(24):12198-12208 (2001)). However, linear sequences that include the above epitopes and recombinant HIV-1 envelope with exposed MPER region, fail to induce neutralizing antibodies. Several plausible explanations include epitope variation, masking of epitopes by a glycan shield, and unfavorable entropic barrier contributing to conformational masking of eptiopes (Kwong et al, Nature 420:678 (2002), Wei et al, Nature 422:307 (2003); Burton et al, Nature Immunol. 5:233 (2004)).

Haynes et al, Science 308:1878 (2005) recently discovered that three of the rare HIV-1 broadly neutralizing antibodies (2F5, 4E10, 1b12) are polyspecific and bind to self antigens that include the anionic phospholipid, cardiolipin. Interaction of 2F5 and 4E10 mAbs with membrane lipids were also proposed in crystal structure studies that showed that the highly hydrophobic CDR3 regions of both mAbs made little contact with the peptide and were largely free (Ofek et al, J. Virol. 78:10724 (2004), Cardoso et al, Immunity 22:163 (2005)). This raises the possibility that the current vaccines fail to produce such mAbs due to their potential self-reactivity, which is regulated such that autoreactive B cells are normally deleted or tolerized against HIV-1 envelope. The present invention results at least in part, from studies designed to test this hypothesis.

The instant invention provides an immunization strategy that allows breaks in tolerance. The invention further provides novel immunogens that present the MPER epitopes in a multivalent form.

### SUMMARY OF THE INVENTION

The present invention relates to immunogens that present MPER epitopes in multivalent form, and to methods of using same in immunization regimens.

Objects and advantages of the present invention will be clear from the description that follows.

### BRIEF DESCRIPTION OF THE DRAWINGS

FIG. 1: Broadly neutralizing antibodies (2F5, 4E10) bind to epitopes that lie proximal to the host membrane. Both 2F5 and 4E1 mAbs are IgG3, have long CDR3s, and bind to epitopes that lie within HIV-1 gp41 (aa 660-683) MPER in a two step conformational change model.

FIG. 2: Peptide sequences used in the generation of B cell tetramers. The nominal epitopes of mAbs 2F5 and 4E10 binding epitopes include sequences ELDKWAS and WFNITNW, respectively. The V3 sequences from gp120 are from clade B. V3 sequences from any HIV-1 clade (e.g., clades A, C, D, E, F, G, H, I) can be used, as well as group M and subtype consensus V3 sequences (Gaschen et al, Science 296:2354 (2002); Los Alamos National Laboratory HIV Sequence Database). Scrambled (Scr) sequences are used controls.

FIGS. 3A and 3B: FIG. 3A. Schematic of B cell tetramers binding to B cell surface immunoglobulin. FIG. 3B: Schematic of an individual tetramer.

FIGS. 4A and 4B: FIG. 4A. 5A9 murine hybridoma B cells that bind the 2F5 gp41 peptide were tested for their ability to bind to the 2F5 tetramer or the control 2F5 scrambled tetramer (top panels). When control hybridoma cells were spiked with 10% (middle panels) or 1% (lower panels) 5A9 hybridoma cells, the 2F5 but not the scrambled tetramer correctly identified the spiked 5A9 B cells. FIG. 4B. The same experiment as in FIG. 4A but with an anti-Ig/tetramer double stain. Results are the same as in FIG. 4A.

FIG. 5: Binding of tetramers to antibody coated beads. The shaded curve shows the binding of the 2F5-epitope tetramer to P3X63 coated beads, the solid line shows the same tetramer binding to beads coated with 2F5. The dashed line is the binding of a scrambled 2F5-epitope tetramer to 2F5 coated beads.

FIGS. 6A-6F: Binding of chromophore labeled tetramers to 4E10 antibody coated beads. In all panels, shaded curves show binding to control Ig-coated beads and solid lines show binding to 4E10 coated beads. FIG. 6A. 2F5-epitope tetramers labeled with APC. FIG. 6B. 2F5-epitope tetramers labeled with PE-AF680. FIG. 6C. V3-epitope tetramers labeled with PE-AF750. FIG. 6D. Scrambled 2F5-epitope tetramers labeled with APC. FIG. 6E. Scrambled 2F5-epitope tetramers labeled with PE-AF680. FIG. 6F. Scrambled V3-epitope tetramers labeled with PE-AF750. Thus, 4E10 mAb binds to phycoerythrin on any tetramer-SA complex.

FIG. 7: Structure of phycoerythrin with chromopore on the molecule surface. (Contreras-Martel, Acta Cryst. 161D57:52-60 (2001).)

FIG. 8: Similarities in tryptophan ring, the chromophore and hemoglobin phycoerythrin ring structures.

FIG. 9: 2F5 tetramers identify more B cells in MRL lpr(−1−) mice than in wildtype BaLB/C mice in B1 B cells.

FIG. 10: 2F5 tetramers identify more B cells in MRL lpr(−1−) mice than in wildtype Balb/C mice in B2 B cells.

FIG. 11: Oligomannose to which broadly neutralizing antibody 2G12 binds. (Poshov et al, Glycobiology 15:994-1011 (2005).)

FIG. 12: Aptamers for the 2G12 epitope. In vitro selection methods were utilized to obtain 2′F pyrimidine RNA aptamers to the HIV neutralizing antibody 2G12. A complex library of ˜10¹⁴ different RNA molecules, which possess distinct secondary and tertiary structures, was bound to 2G12. Those RNAs that bind were separated by a nitrocellulose partitioning scheme, reamplified by RT-PCR with primers specific for the fixed regions, and then transcribed. The process was repeated several times to obtain four RNA aptamers specific to 2G12.

FIG. 13: 2G12 aptamer binds to gp120 with a Kd of about 250 to about 500 nM.

### DETAILED DESCRIPTION OF THE INVENTION

This present invention relates generally to immunization strategies and protocols for the generation of anti-HIV-1 neutralizing antibodies and for the detection of antigen-specific B cell responses. In one embodiment, the invention relates to synthetic biotin-streptavidin conjugates containing HIV-1 epitopes, and to compositions comprising same. In a further embodiment, the invention relates to a method of generating broadly neutralizing antibodies against HIV-1 in a patient comprising administering such conjugates. In yet another embodiment, the invention relates to a method of monitoring immune responses to HIV-1 immunogens using such conjugates as diagnostic reagents to detect specific B cell responses.

### Immunogen Design

Conjugates of the invention are B cell tetramers that can comprise nominal epitope peptides of two broadly neutralizing antibodies that bind to the MPER of HIV-1 gp41 as well as the V3 region of HIV gp120. Alternatively, the tetramers can comprise carbohydrate antigens of gp120 conjugated to biotin. (B cell tetramers, albeit different from those disclosed here, have been used previously to identify antigen-specific B cell populations (see, for example, Newman et al, J. Immunol. Methods 272:177-187 (2003), Rice et al, Proc. Natl. Acad. Sci. USA 102:1608-1613 (2005) and Scibelli et al, Vaccine 23:1900 (2005)).

Peptide sequences that include the nominal epitopes of mAbs 2F5 and 4E10, respectively, can be linked to any of a variety of spacer molecules well known in the art using standard peptide chemistry (FIG. 2). Two specific spacers that have been used successfully are shown in FIG. 2 (e.g. 3-5 G's and —(CH₂)₅—). As shown in FIG. 2, biotin can be placed at either the N terminal or C terminal end of the peptide. Such constructs provide unconstrained access of mAbs to their respective epitopes.

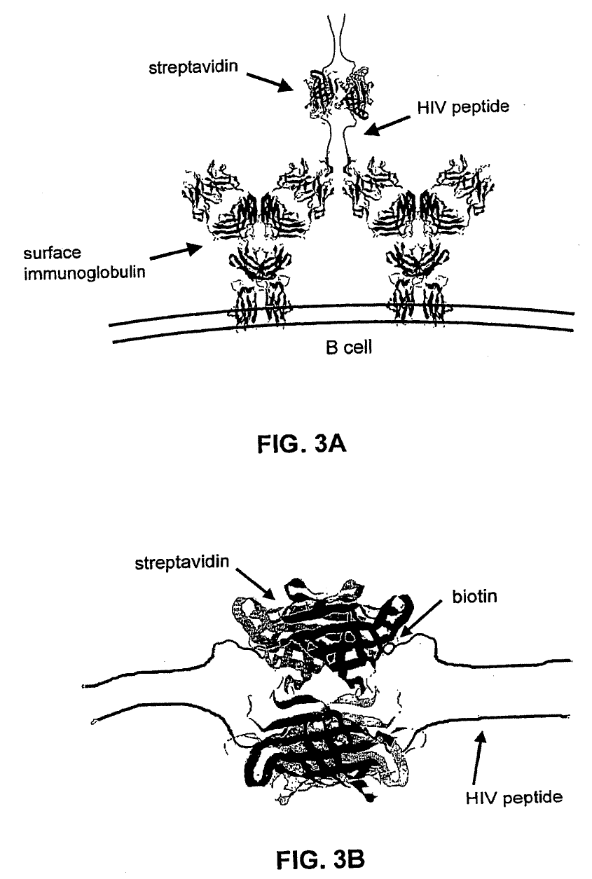

Tetramers of the invention can be prepared, for example, by first dissolving the peptide in a suitable medium such as phosphate buffered saline containing 0.1% w/v of sodium azide. The concentration of the peptide can be adjusted to, for example, 200 μM. Streptavidin labeled, for example, with a desired fluorochrome can be prepared to a concentration of, for example, 6 μM. Equal volumes of the peptide solution and the solution of streptavidin can be mixed and incubated at, for example, 4° C. for 4-16 hours. The reaction can then be returned to room temperature and the unbound peptide removed from the tetramer, for example, by the use of gel filtration chromatography. Gel filtration medium with a molecular weight cutoff of, for example, 40,000 can be equilibrated with phosphate buffered saline with 0.1% sodium azide. The reaction mixture can be passed through the gel filtration medium to obtain tetramer free unbound peptide. The tetramer preparation can then be analyzed for overall protein content by standard assays and the specific binding of the tetramer verified using, for example, beads coated with the antibodies of interest and cell lines expressing those antibodies (FIGS. 3A and 3B).

### Method of Quality Control and Analysis of Specificity of the Constructed HIV-1 Tetramers

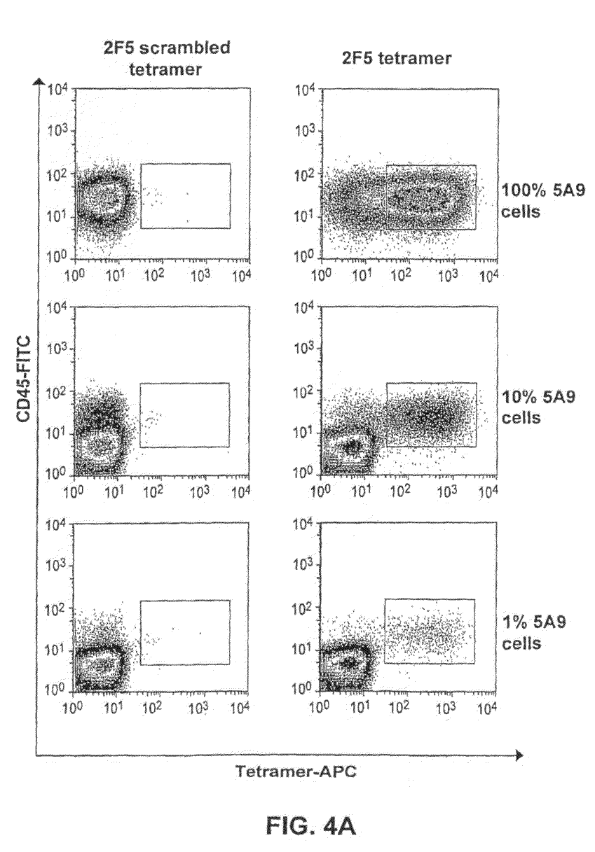

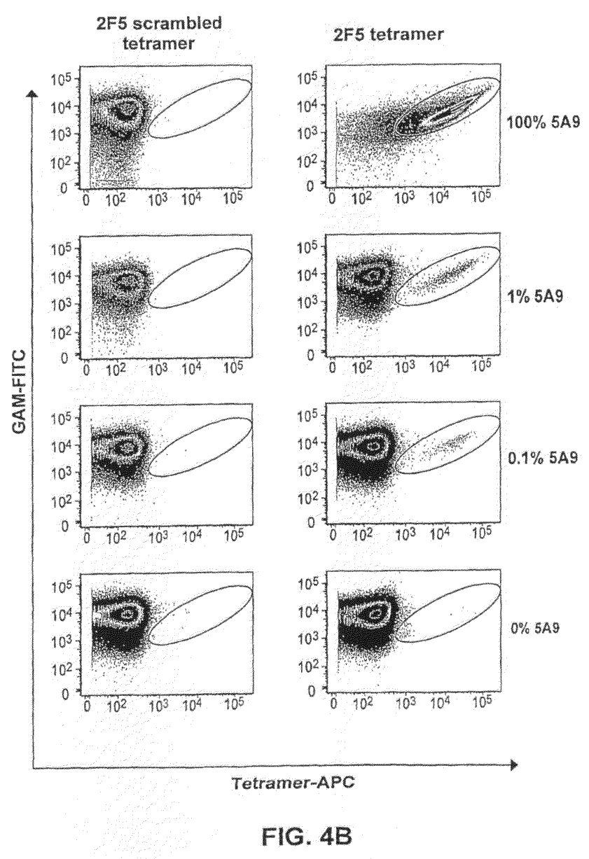

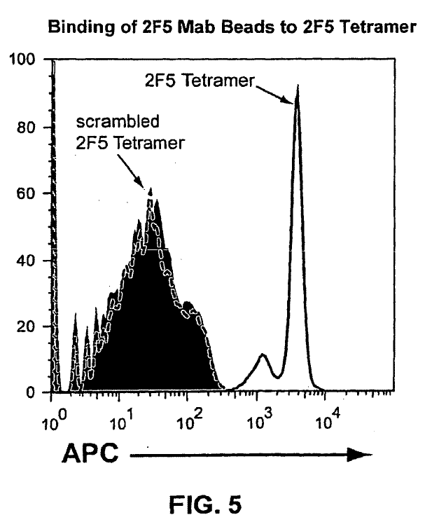

The specificity of the tetramers can be determined using a panel of murine hybridoma cell lines that produce either antibodies that react with the 2F5 epitope (5A9), the 4E10 epitope of HIV gp41 (10B12) or the V3 region of HIV gp120 (7B9 or F39F). Using these cell lines, the B cell tetramer can be bound to the cell line and assayed for binding by, for example, flow cytometry (FIGS. 4A and 4B). Alternatively, the 2F5, 4E10 anti-MPER and 7B9 anti-V3 mAbs can be conjugated to, for example, a 3 μM bead, and the specificity of tetramer binding to the beads determined (FIG. 5).

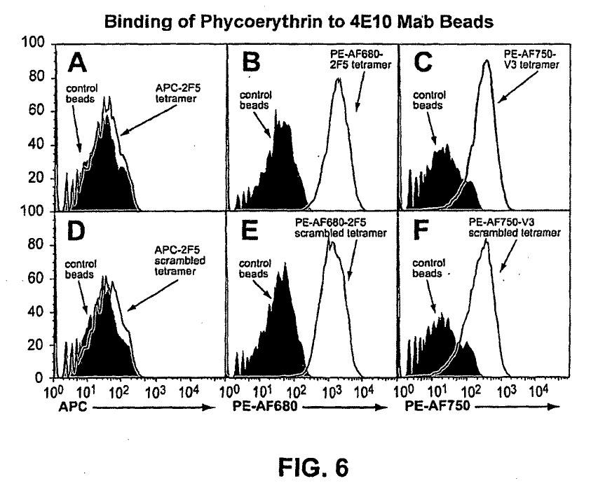

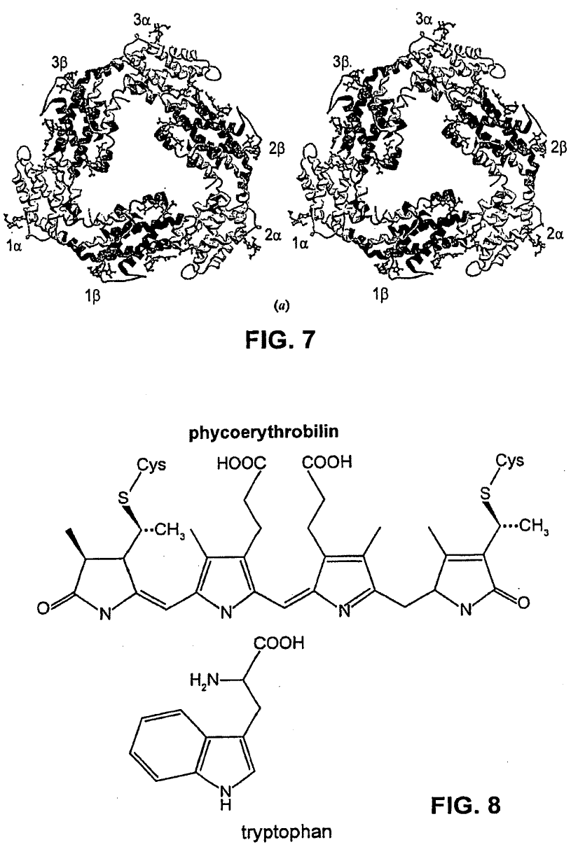

Studies conducted have shown that a mimetope of the MPER 4E10 region is phycoerythrin, in that 4E10 mAb coated beads bound tetramer labeled with phycoerythrin but not allophycocyanin (APC) (FIG. 6). The likely binding site on phycoerythrin is the ring structure of the surface chromophore of the PE molecule (FIG. 7). This structure is similar to the tryptophan ring that is associated with 4E10 binding to the gp41 MPER region (FIG. 8).

### Identification of B Cell Precursors Capable of Making 2F5 Antibodies in Normal and Autoimmune Mice.

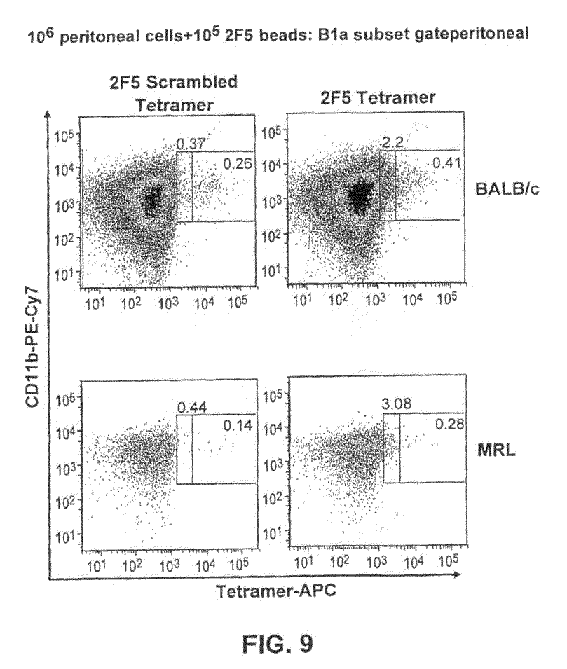

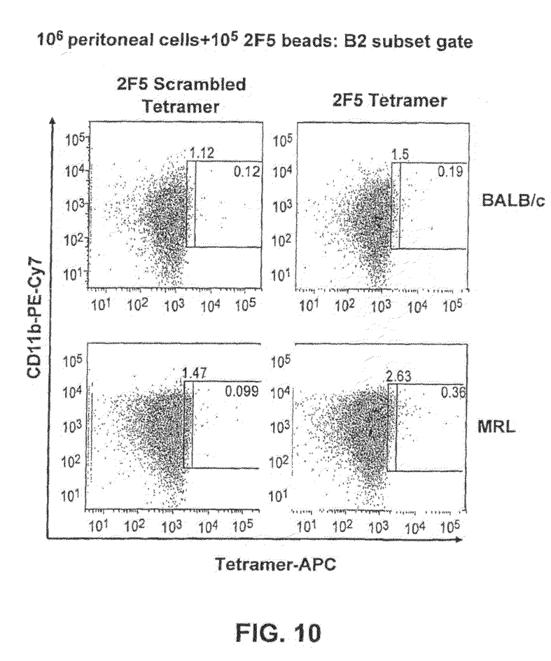

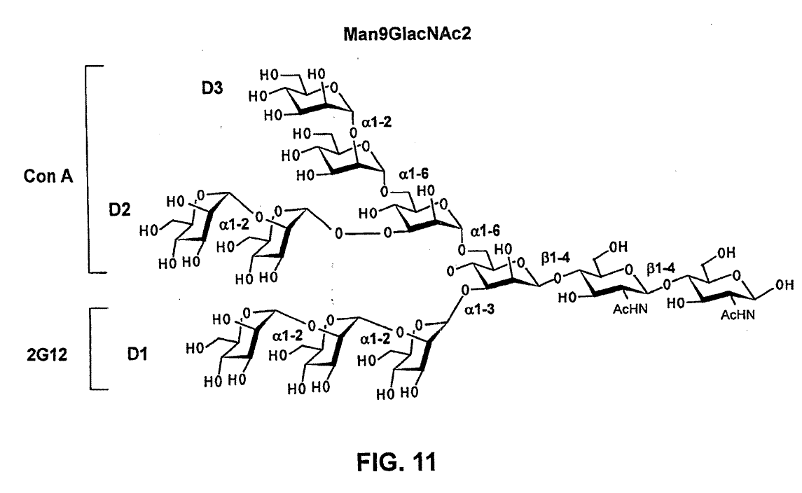

Since characteristics of 2F5 and 4E10 MAbs demonstrate that they are autoantibodies and, therefore, are likely subjected to B cell tolerance mechanisms, elevated levels of MPER B cell precursors can be expected in autoimmune mice and humans. FIGS. 9 and 10 show that using the 2F5 vs 2F5 scrambled tetramers, it is possible to demonstrate elevated levels of 2F5 gp41 epitope reactive B cells in MRL-lpr(−1−) (autoimmune) mice that are both in the B1 (innate B cell) and the B2 (adaptive B cell) pools of B cells.

Identification of B Cell Precursors Capable of Making 2G12 like Antibodies in Normal and Autoimmune Mice.

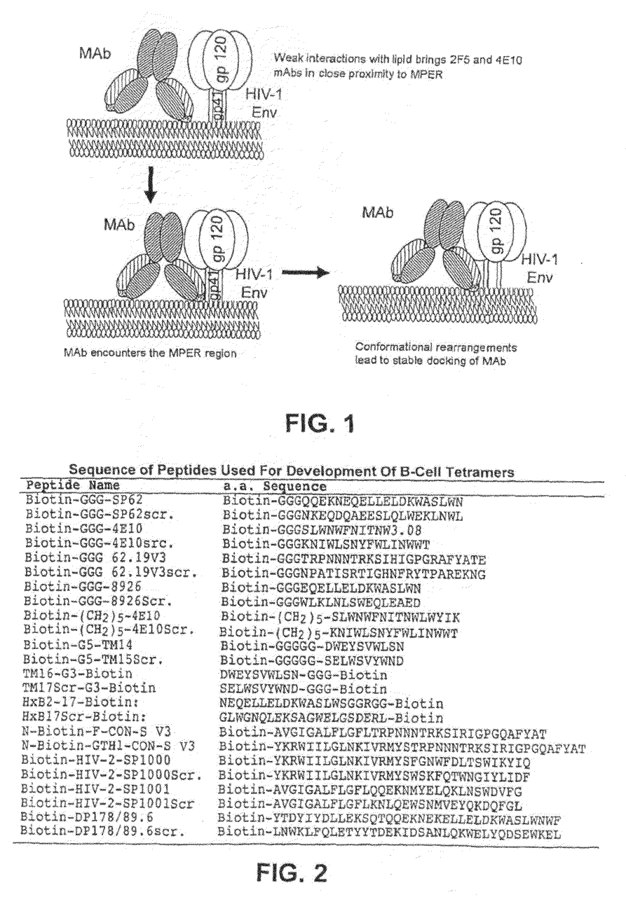

The broadly neutralizing antibody 2G12 reacts with an oligomannose residue on the surface of HIV gp120 (Calarese et al, PNAS USA 102:13372-7 (2005)) (FIG. 1). This sugar can be conjugated to biotin and a tetramer made of the sugar for identification of B cell precursors making 2G12-like antibodies.

Chromophore-conjugated tetramers can be used, for example, in flow cytometric assays as a reagent for the detection of HIV-1 anti-MPER specific B cell responses in animals and humans immunized with HIV-1 Env proteins that present exposed MPER or other HIV env regions. Thus, these reagents can be used to study peripheral blood B cells to determine the effectiveness of immunization for anti-MPER antibody induction by measuring the number of circulating memory B cells after immunization.

### Immunization Strategy

The immunization strategy of the invention incorporates a regimen that allows temporary breaks in tolerance. An exemplary protocol involves the use of oCpGs, the TLR9 ligand that has been used to break tolerance for the production of anti-dsDNA antibodies in mice (Tran et al, Clin. Immunol. 109(3):278-287 (2003)). In accordance with this approach, peptide-liposome conjugates can be mixed (e.g., 1:1) with the adjuvant, e.g., Emulsigen plus oCpG. The Emulsigen adjuvant can be prepared, for example, by mixing 375 μL of Emulsigen, 250 μL of oCpG and 625 μL of saline. Guinea pig can be immunized on a 21-day interval with 250 μg of either peptide monomer or peptide tetramer. The tetramer will have enhanced apparent affinity to B cell receptor+B cells because of enhanced avidity, and will, therefore, trigger B cells in an enhanced manner compared to monomer of the nominal HIV epitope.

Another suitable protocol involves the use of strategies to temporarily deplete T regulatory cells using, for example, anti-CD25 mAbs, or protein or DNAs expressing GITR ligand (Stone et al, J. Virol. 80:1762-72 (2006)), or CD40 Ligand (Stone et al, J. Virol. 80:1762-72 (2006)). (See also U.S. application Ser. No. 11/302,505.)

A further protocol for breaking tolerance involves conjugating the immunogen with heterologous proteins such as phycoerythrin, keyhole limpet hemocyanin or ovalbumin (Scibelli et al., Vaccine 23:1900 (2005)).

Alternatively, immunization can be IV, intranasal, subcutaneous, intraperitoneal, intravaginal or intrarectal with tetramers formulated in adjuvants such as oCpGs, TLR4 agonists, or TLR7 agonists that facilitate robust antibody responses, as well as DNAs expressing GITR ligand and/or CD40 ligand.

Interfering RNAs (iRNAs) can also be used to inhibit the tristetraproline gene that encodes a protein that induces the degradation of the TNF α gene and protein (Taylor et al, Immunity 4:445 (1996); Carballo et al, J. Clin. Invest. 100:986 (1997)). Deletion of the TTP gene leads to unimpeded TNFα production and autoimmunity. Temporary interruption of the degradation of the TTP gene will lead to enhanced immunity to a vaccine. Thus administration of soluble iRNAs themselves or encoded in a DNA immunization can be used as an adjuvant to administered with B cell tetramers.

Given that phosphatidylethanol amine (PE) binds to the broadly neutralizing antibody 4E10 and is a mimetope for the gp41 MPER neutralizing epitope, PE itself can be administered either alone or with the 4E10 B cell tetramer as an immunogen to induce anti-MPER neutralizing antibodies. Advantageously, the 4E10 tetramer containing streptavidin conjugated to PE can be used as a chimeric immunogen containing 4 copies of the nominal MPER epitope and PE on the surface of Streptavidin. Finally, tetramers comprising the nominal epitopes of the MPER region, the V3 region and the carbohydrate oligomannoses that bind to the neutralizing antibody 2G12 can be combined for a multivalent immunogen for protection against HIV infection.

### Construction of B Cell Tetramers Using RNA Aptamer.

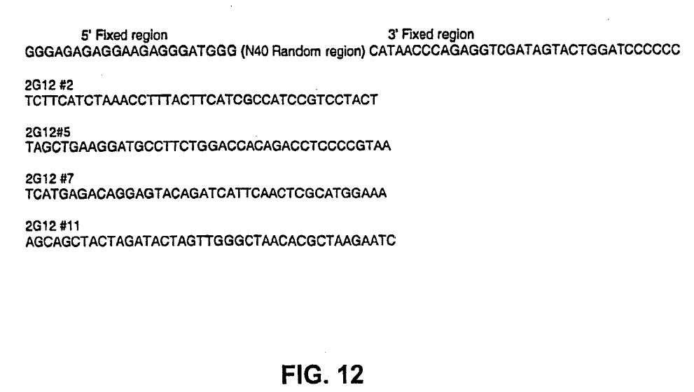

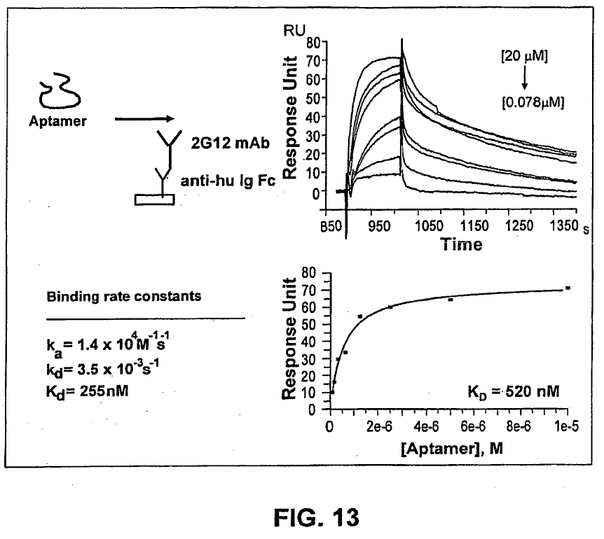

An alternative method of construction of Tetramers for identifying broadly neutralizing antibody producing cells, and for inducing protective antibodies, is the use of RNA aptamer mimetopes that are biotinylated and can be tetramerized with streptavadin. This can be done for any HIV 1 epitope (see Becker et al, Thromb. Haemost. 93(6):1014-20 (2005), Nimjee et al, Annu., Rev. Med. 56:555-83 (2005), Santulli-Marotto et al, Cancer Res. 63(21):7483 (2003) for general aptamer methods and rationale (see also U.S. Pat. Nos. 5,270,163, 5,559,877, 5,696,249, 6,110,900 and 6,933,116). Aptamers for the 2G12 epitope have been prepared (FIG. 12). In the case of the 2G12 aptamer, it binds to HIV gp120 with a Kd of about 250 to about 500 nM (FIG. 13). Thus, aptamers derivatized with biotin and made into tetramers, derivatized with other materials, such as poly L lysine, to create multimers, can be used either alone or with other tetramers as immunogens. Aptamers can be formulated with any of a variety of adjuvants for enhanced immunogenicity.

All documents and other information sources cited above are hereby incorporated in their entirety by reference.

What is claimed is:

1. A conjugate comprising:
i) epitope peptides of two neutralizing antibodies that bind to the membrane external proximal region (MPER) of HIV-1 gp41 and the V3 region of HIV 120, or
ii) carbohydrate antigens of gp120 conjugated to biotin.

2. The conjugate according to claim 1 wherein said conjugate comprises said epitope peptides and wherein said neutralizing antibodies are 2F5 and 4E10.

3. The conjugate according to claim 1 wherein said epitopes are linked to a spacer molecule.

4. The conjugate according to claim 3 wherein said spacer molecule comprises 3-5 G's or —(CH₂)₅—.

5. The conjugate according to claim 1 wherein biotin is linked to the N terminal ends of said peptides.

6. The conjugate according to claim 1 wherein biotin is linked to the C terminal ends of said peptides.

7. The conjugate according to claim 1 wherein said conjugate comprises said epitope peptides and wherein said epitope peptides are selected from the group consisting of the epitope peptides set forth in FIG. 2.

8. The conjugate according to claim 1 wherein said conjugate is a B cell tetramer comprising peptides selected from the group consisting of the peptides set forth in FIG. 2.

9. The conjugate according to claim 1 wherein said conjugate is conjugated with phycoerythrin, keyhole limpet hemocyanin or ovalbumin.

10. A method of inducing broadly neutralizing antibodies against HIV in a patient in need thereof comprising administering to said patient an amount of the conjugate according to claim 1 sufficient to effect said induction.

11. The method according to claim 10 wherein said patient is a human.

12. The method according to claim 10 further comprising administering to said patient an adjuvant.

13. The method according to claim 12 wherein said adjuvant comprises Emulsign, oCpGs, a TLR4 against, a TLR7 agonist, or iRNAs that inhibit the tristetrapraline gene.

14. The method according to claim 10 wherein said method further comprises administering to said patient an agent that depletes T regulatory cells.

15. The method according to claim 14 wherein said agent comprises anti-CD25 antibodies, a GITR ligand or a CD40 ligand.

16. A composition comprising tetramers comprising nominal epitopes of the MPER region, the V3 region and carbohydrate oligomannoses that bind to 2G12.

17. A method of inducing neutralizing antibodies against HIV in a patient in need thereof comprising administering to said patient an amount of the composition according to claim 16 sufficient to effect said induction.

18. A composition comprising a biotinylated aptamer for an HIV epitope tetramerized with streptavadin.

19. A method of inducing an immune response in a patient in need thereof comprising administering to said patient an amount of the composition according to claim 18 sufficient to effect said induction.
